(19) United States
(12) Patent Application Publication
(10) Pub. No.: US 2010/0329996 A1
(43) Pub. Date: Dec. 30, 2010

(54) Novel Combination of Therapeutic Agents

(75) Inventor: Dramane Ibrahim Laine, King of Prussia, PA (US)

Correspondence Address: GlaxoSmithKline;GLOBAL PATENTS -US, UW2220, P. O. BOX 1539, KING OF PRUSSIA, PA, 19406-0939, US

(73) Assignee: Glaxo Group Limited

(21) Appl. No.: 12/677,160

(22) PCT Filed: Sep. 12, 2008
(86) PCT No.: PCT/US08/76122

§ 371 Date: Mar. 9, 2010

Related U.S. Application Data
(60) Provisional application No. 60/971,646, filed on Sep. 12, 2007.
(60) Provisional application No. 61/092,917, filed on Aug. 29, 2008.

(51) Int. Cl. A61K 9/12; A61K 31/7016; A61K 31/569; A61K 31/4375; A61P 11/00; A61P 29/00; A61P 11/06

(52) U.S. Cl. 424/45; 424/43; 514/53; 514/171; 514/299

(57) ABSTRACT

Novel combinations of a muscarinic acetylcholine receptor antagonist and a beta 2 agonist and/or a corticosteroid for inhaled administration via the nose or mouth, and methods of using them are provided herein.

### FIELD OF THE INVENTION

This invention relates to pharmaceutical products and compositions for use in the treatment of asthma and related disorders, such as the treatment of chronic obstructive pulmonary disease (COPD).

More particularly this invention relates to the combination of a muscarinic antagonist with at least one additional therapeutic agent, and their use in treating M₃ muscarinic acetylcholine receptor mediated diseases, and other therapeutic indications for which the second therapeutic agent is known.

### BACKGROUND OF THE INVENTION

Selective β₂-adrenoreceptor agonists have been used in the prophylaxis and treatment of clinical conditions for which a bronchodilating agent has been indicated. Such conditions include diseases associated with [reversible] airways obstruction such as asthma, chronic obstructive pulmonary diseases (COPD) (e.g. chronic and wheezy bronchitis, emphysema), respiratory tract infection and upper respiratory tract disease (e.g. rhinitis, including seasonal and allergic rhinitis).

In particular, the treatment of asthma and other related disorders has been with these beta-2 adrenergic receptor agonists (beta-2 agonists) as they provide a bronchodilator effect to the patient, resulting in relief from the symptoms of breathlessness. Within the beta-2 agonist class there are presently available short acting compounds for immediate relief, such as salbutamol, biltolterol, pirbuterol and terbutaline. There are also longer acting compounds commercially available, such as salmeterol and formoterol. Although salmeterol and the other commercially available β₂-adrenoreceptor agonists are effective bronchodilators, in general their duration of action in human subjects is around 12 hours, hence twice daily dosing is often required.

While the beta-2 agonists provide for symptomatic relief of bronchoconstriction in patients, another component of asthma, i.e. inflammation, often requires separate treatment. Typically, this treatment has been with a steroid. Currently available corticosteroids for use include beclomethasone, budesonide, flunisolide, fluticasone propionate, mometasone furoate and triamcinolone.

Over the last two decades, inhaled anticholinergics have become well established as safe and effective bronchodilators for the treatment of COPD. Treatment with anticholinergics significantly improves FEV₁, (forced expiration of volume over 1 second) resting and dynamic lung hyperinflation, symptoms, and exercise capacity and reduces COPD exacerbations. Currently, only a few inhaled anticholinergic bronchodilators are available: the short-acting ipratropium bromide (ipratropium; dosed four-times-a-day) and oxitropium bromide, and the long-acting tiotropium bromide (tiotropium; dosed once-daily).

International COPD treatment guidelines [American Thoracic Society (ATS) and European Respiratory Society (ERS) Standards for the Diagnosis and Management of Patients with COPD and the Global Initiative for Chronic Obstructive Lung Disease (GOLD)] recommend an incremental approach to treatment as the disease state worsens, involving the use of combinations of drug classes with different or complementary mechanisms of action. For patients with moderate disease, regular treatment with one or more long-acting bronchodilators is recommended, progressing to the addition of an inhaled corticosteroid for more severe COPD.

Improved efficacy with similar safety of the individual components provides the rationale for development of a long-acting anticholinergic and a beta₂-agonist (dual) fixed dose combination product, and a long-acting anticholinergic, beta₂-agonist, and an inhaled corticosteroid (triple) fixed dose combination product for the treatment of COPD. Further, a long-acting anticholinergic, and an inhaled corticosteroid (dual) fixed dose combination product for treatment of asthma and other related disorders is also needed.

In order to achieve better patient compliance with treatment regimens, once daily or twice daily dosing with one or more agents at the same time (simultaneously, or concurrently) or with consecutive dosing (sequential) are advantageous. These various combinations would allow for simplification of common polypharmacy regimens and may also improve treatment adherence.

Some combination products are known, e.g. an inhalation combination medication of fluticasone propionate and salmeterol, the combination being provided in one easy-to-use device. This combination product provides simultaneous treatment of airway constriction by means of the beta-2 agonist (salmeterol), and treatment of inflammation by means of the steroid (fluticasone propionate).

A combination of ipratropium bromide and salbutamol is also known. This combination therapy provides an anti-cholinergic (ipratropium bromide) to reduce the bronchial secretions and a beta-2 agonist (salbutamol) to reduce constriction. Other combinations include ipratropium and salbutamol (WO 01/76601) and tiotropium and formoterol (WO 00/47200).

It would be highly desirable, however, to provide a combination therapy suitable to reduce bronchial inflammation, bronchial constriction and bronchial secretions in a single product or dosage form. It would also be desirable to provide such a combination product or composition in a form whereby the correct dosage of the various components is easily and safely administered. The present invention is directed to such a need in the marketplace.

### SUMMARY OF THE INVENTION

This invention provides for the novel combination of a pharmaceutically acceptable anion of (Endo)-3-(2-cyano-2,2-diphenyl-ethyl)-8,8-dimethyl-8-azonia-bicyclo[3.2.1]octane (compound (I)); and at least one of the following other therapeutic agents selected from the group consisting of salmeterol xinafoate, and fluticasone propionate; and wherein each of the therapeutic agents are optionally present in enantiomerically pure form or as a racemic mixture, as a pharmaceutical product.

This invention also provides for use of the product in the manufacture of a medicament for the prophylaxis or treatment of conditions for which administration of one or more of the therapeutic compounds is indicated.

In one embodiment the use is for the treatment of inflammatory or respiratory tract diseases, by simultaneous or successive administration of the first therapeutic agent and at least one other therapeutic agent.

In another embodiment the use is for the manufacture of a medicament for the treatment of asthma and/or chronic obstructive pulmonary disease (COPD), by simultaneous or successive administration of a first therapeutic agent and at least one other therapeutic agent.

Another embodiment of the invention is a method for the prophylaxis or treatment of inflammatory or respiratory tract diseases, comprising administering either sequentially or simultaneously, to a patient in need thereof, a pharmaceutical product comprising a first therapeutic agent which is a pharmaceutically acceptable anion of (Endo)-3-(2-cyano-2,2-diphenyl-ethyl)-8,8-dimethyl-8-azonia-bicyclo[3.2.1]octane (compound (I)); and at least one other therapeutic agent selected from the group consisting of salmeterol xinafoate, and fluticasone propionate; and wherein each of the therapeutic agents are optionally present in enantiomerically pure form or as a racemic mixture, as a pharmaceutical product.

In one embodiment of the invention the inflammatory or respiratory tract disease is selected from the group consisting of chronic obstructive pulmonary disease, chronic bronchitis, asthma, chronic respiratory obstruction, pulmonary fibrosis, pulmonary emphysema and allergic rhinitis.

In another embodiment of the invention the pharmaceutical product may be used for the treatment of the inflammatory or respiratory tract disease, and more specifically the treatment of asthma and/or chronic obstructive pulmonary disease (COPD), by either simultaneous or successive administration of the first therapeutic agent and the at least one other therapeutic agent. It is recognized that the first therapeutic agent may be given simultaneously with only one of the other therapeutic agents, and the second other therapeutic agent my then be given successively thereafter or all three may be administered concurrently together.

### DETAILED DESCRIPTION OF THE INVENTION

The present invention is directed to a pharmaceutical product comprising

a) a first therapeutic agent selected from a compound of the formula

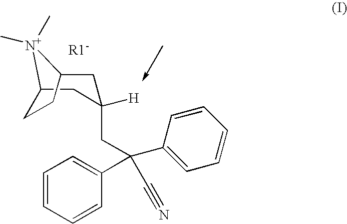

wherein:
the H atom indicated is in the exo position;
R1⁻ represents a pharmaceutically acceptable anion associated with the positive charge of the N atom; and

b) at least one of the following other therapeutic agents selected from the group consisting of salmeterol xinafoate, and fluticasone propionate; and wherein each of the therapeutic agents are optionally present in enantiomerically pure form or as a racemic mixture. The at least one other agent is referred to herein as Compound (II). If there are 2 additional agents, the third compound is referred to herein as Compound (III), etc.

In one embodiment of the invention the compound of Formula (I), the pharmaceutically anion is selected from chloride, bromide, iodide, sulfate, benzene sulfonate or toluene sulfonate.

In one embodiment of the invention, the anion is bromide and the compound of Formula (I) is (Endo)-3-(2-cyano-2,2-diphenyl-ethyl)-8,8-dimethyl-8-azonia-bicyclo[3.2.1]octane bromide. This specific compound is alternatively referred to herein as the bromide compound.

The amount of a compound of Formula (I), and specifically (Endo)-3-(2-cyano-2,2-diphenyl-ethyl)-8,8-dimethyl-8-azonia-bicyclo[3.2.1]octane bromide, required to achieve a therapeutic effect will, of course, vary with the route of administration, the subject under treatment, and the particular disorder or disease being treated. Suitably, the route of administration is by inhalation via the nose or by oral inhalation therapy.

Compounds of Formula (I), and specifically the (Endo)-3-(2-cyano-2,2-diphenyl-ethyl)-8,8-dimethyl-8-azonia-bicyclo[3.2.1]octane bromide (the bromide compound) may be administered by inhalation at a dose of from about 10 mcg to about 200 mcg/daily, and if necessary in divided doses.

The bromide compound has been found to be dose dependent. By this it is meant that the bromide compound has now been shown to have a slow “off-rate” at the muscarinic acetylcholine (Ach) receptor. This is known to be predictive of a long duration of action. Therefore, while a twice daily dosing at 10-100 mcg/dose may be used, if the dose is increased, such as to 100-200 mcg/dose, once daily dosing may be a suitable alternative.

Importantly, the bromide compound has been found to have a 10-fold selectivity for the M1 and M3 receptors over the M2 receptors, and the bromide compound has also been found to be a partially reversible antagonist.

Fluticasone propionate is generally administered via the inhaled route to a human either (a) at a dose of 250 micrograms once per day or (b) at a dose of 50 to 250 micrograms twice per day. It is recognized that the amount of fluticasone propionate may be increased in daily amounts as deemed necessary, such as to an amount of 500 micrograms administered once per day.

Salmeterol or a pharmaceutically acceptable salt thereof, e.g. salmeterol xinafoate, is generally administered to humans at an inhaled dose of 25 to 50 micrograms twice per day (measured as the free base).

The presence in the market place of a therapeutic three-in-one combination comprising a salmeterol, and fluticasone propionate (Advair/Seritide) in combination with a specific anti-cholinergic agent would provide for a hereinto unavailable control of bronchoconstriction, inflammation and mucous secretions of airways. A three-in-one combination therapy as provided for by the present invention would also have an extremely patient-friendly aspect resulting in maximal patient compliance and better control of asthma than the known combinations or single therapies.

The present invention therefore provides for a pharmaceutical product comprising a combination of therapeutic agents, for simultaneous, separate or sequential use in the treatment of conditions for which administration of one or more of the therapeutic agents are indicated.

The formulations for use herein include various forms of inhalation administrations (including fine particle dusts or mists which may be generated by means of various types of metered dose pressurised aerosols, nebulisers or insufflators), as will be further described herein below.

Dry powder compositions for topical delivery to the lung by inhalation may, for example, be presented in capsules and cartridges of for example gelatine, or blisters of for example laminated aluminium foil, for use in an inhaler or insufflator. Powder blend formulations generally contain a powder mix for inhalation of the compound of the invention and a suitable powder base (carrier/diluent/excipient substance) such as mono-, di or poly-saccharides (e.g. lactose or starch). Use of lactose is preferred.

Dry powder compositions may also include, in addition to the drug and carrier, a further excipient (e.g. a ternary agent) such as a sugar ester, for example cellobiose octaacetate, calcium stearate or magnesium stearate. Alternatively, the compound of the invention may be presented without excipients. For the avoidance of doubt use of the term ‘composition’ herein refers to the therapeutic compounds either with or without excipients or carriers.

Each capsule or cartridge may generally contain between 10 μg-200 μg of the bromide compound, optionally in combination with the other therapeutically active agent(s). Alternatively, the compound of the invention may be presented without excipients. Packaging of the formulation may be suitable for unit dose or multi-dose delivery. If the pharmaceutical product contains two additional active agents, e.g. both salmeterol and fluticasone, the bromide compound may be in admixture with the salmeterol and the fluticasone may be in its own capsule or the bromide compound may be in admixture with the fluticasone and the salmeterol may be in its own capsule or cartridge. Yet in another alternative embodiment, the bromide compound may be in its own capsule or cartridge, etc. and the salmeterol and fluticasone are together in their own capsules or cartridge for administration to the patient. Yet another embodiment if the pharmaceutical product contains both additional active agents it is possible to have all three actives in their own cartridge or capsule, with or without additional excipients as deemed necessary.

Optionally for these dry powder inhalable products, a composition suitable for inhaled administration may be incorporated into a plurality of sealed dose containers (e.g. containing the dry powder compositions) provided on medicament pack(s) mounted inside a suitable inhalation device. The containers may be rupturable, peelable or otherwise openable one-at-a-time and the doses of the dry powder composition administered by inhalation on a mouthpiece of the inhalation device, as known in the art. The medicament pack may take a number of different forms, for instance a disk-shape or an elongate strip. Representative inhalation devices are the DISKHALER™ and DISKUS™ devices, marketed by GlaxoSmithKline. The DISKUS™ inhalation device is, for example, described in GB 2242134A.

A dry powder inhalable composition, e.g. the pharmaceutical product, may also be provided as a bulk reservoir in an inhalation device, the device then being provided with a metering mechanism for metering a dose of the composition from the reservoir to an inhalation channel where the metered dose is able to be inhaled by a patient inhaling at a mouthpiece of the device. Exemplary marketed devices of this type are TURBUHALER™ of AstraZeneca, TWISTHALER™ of Schering and CLICKHALER™ of Innovata.

A further delivery method for a dry powder inhalable composition (pharmaceutical product) is for metered doses of the composition to be provided in capsules (one dose per capsule) which are then loaded into an inhalation device, typically by the patient on demand. The device has means to rupture, pierce or otherwise open the capsule so that the dose is able to be entrained into the patient's lung when they inhale at the device mouthpiece. As marketed examples of such devices there may be mentioned ROTAHALER™ of GlaxoSmithKline and HANDIHALER™ of Boehringer Ingelheim. It is recognized that while ‘a capsule’ is referred to herein, it is also intended to include two or three capsule inhalation to accommodate the aforementioned combinations of active agents.

A dry powder composition may also be presented in a delivery device which permits separate containment of the bromide compound alone or admixed with salmeterol or fluticasone as one or two agents together. So for example, the individual compounds of this combination are administrable simultaneously but are stored separately (or wholly or partly stored separately for triple combinations), e.g. in separate pharmaceutical compositions, for example as described in WO 03/061743 A1 and/or WO 2007/012871 A1. A further device that permits separate containment of different compounds is DUOHALER™ of Innovata.

Spray compositions for inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the pharmaceutical product and a suitable propellant such as a fluorocarbon or hydrogen-containing chlorofluorocarbon or mixtures thereof, particularly hydrofluoroalkanes, especially 1,1,1,2-tetrafluoroethane, 1,1,1,2,3,3,3-heptafluoro-n-propane or a mixture thereof. The aerosol composition may optionally contain additional formulation excipients well known in the art such as surfactants e.g. oleic acid, lecithin or an oligolactic acid derivative e.g. as described in WO 94/21229 and WO 98/34596 and cosolvents e.g. ethanol.

There is thus provided as a further aspect of the invention a pharmaceutical aerosol formulation product comprising compound (I), and specifically (Endo)-3-(2-cyano-2,2-diphenyl-ethyl)-8,8-dimethyl-8-azonia-bicyclo[3.2.1]octane bromide, and at least one or salmeterol or fluticasone with a fluorocarbon or hydrogen-containing chlorofluorocarbon as propellant, optionally in combination with a surfactant and/or a co-solvent.

According to another aspect of the invention, there is provided a pharmaceutical aerosol formulation wherein the propellant is selected from 1,1,1,2-tetrafluoroethane, 1,1,1,2,3,3,3-heptafluoro-n-propane and mixtures thereof. Another aspect of the invention is the aerosol formulation solely of compound (I), and specifically (Endo)-3-(2-cyano-2,2-diphenyl-ethyl)-8,8-dimethyl-8-azonia-bicyclo[3.2.1]octane bromide, with a fluorocarbon or hydrogen-containing chlorofluorocarbon as propellant, optionally in combination with a surfactant and/or a co-solvent. In another embodiment of the invention the propellant is selected from 1,1,1,2-tetrafluoroethane, or 1,1,1,2,3,3,3-heptafluoro-n-propane and mixtures thereof.

The formulations of the invention may be buffered by the addition of suitable buffering agents.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient as produced may be size reduced by conventional means e.g. by micronization. The desired fraction may be separated out by air classification or sieving. Preferably, the particles will be crystalline. When an excipient such as lactose is employed, generally, the particle size of the excipient will be much greater than the inhaled medicament within the present invention. When the excipient is lactose it will typically be present as milled lactose, wherein not more than 85% of lactose particles will have a MMD of 60-90 μm and not more than 15% will have a MMD of less than 15 μm.

Magnesium stearate, if present in the formulation, is generally used in an amount of about 0.1 to 2%, e.g. 0.5 to 2%, e.g. 0.75%, 1%, 1.25% or 1.5%. The magnesium stearate will typically have a particle size in the range 1 to 50 μm, and more particularly 1-20 μm, e.g. 1-10 μm. As is well known in the art stearic acid may comprise a mixture of stearic and palmitic acids; small amounts of other acids, e.g. lauric acid, myristic acid and/or arachic acid may also be present. Hence magnesium stearate similarly may comprise a mixture of salts formed with said acids. In general, the proportion of stearic acid present is 0.0 to 100%. Typically the proportion of stearic acid is present in an amount from 60 to 75% with the total proportion of stearic and palmitic acids in an amount from 96-100%.

Addition of magnesium stearate in the formulation with Compound (I) under conditions tested demonstrated some improvement in stabilization of the product on stability.

Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulation may be formulated with an aqueous vehicle with the addition of agents such as acid or alkali, buffer salts, isotonicity adjusting agents or antimicrobials. They may be sterilized by filtration or heating in an autoclave, or presented as a non-sterile product.

It will be appreciated that a subject suffering from a condition such as asthma, may variously from time to time display no overt symptoms of the conditions, or may suffer from periodic attacks during which symptoms are displayed or may experience exacerbations or worsening of the condition. In this context the term ‘treatment’ is intended to encompass prevention of such periodic attacks or exacerbations of the existing condition. Such treatment may be referred to as ‘maintenance treatment’ or ‘maintenance therapy’.

The individual compounds of the pharmaceutical product as described herein may be administered either sequentially or simultaneously in separate or combined pharmaceutical formulations/compositions. Thus the bromide and one or more other therapeutic agents may for example be formulated separately and presented in separate packs or devices, such as for sequential administration, or said individually formulated components may be presented in a single pack or device. Where appropriate, the individual compounds may be admixed within the same formulation, and as such presented as a fixed pharmaceutical combination. In general such formulations will include pharmaceutical carriers or excipients as described hereinafter, but combinations of the compounds without any excipients are also within the ambit of this invention. In one embodiment, the individual compounds will be administered simultaneously in a combined pharmaceutical formulation. Appropriate doses of known therapeutic agents will readily be appreciated by those skilled in the art.

In further aspects the invention therefore provides:

Wherein Compound (I) and Compound (II) are presented separately for sequential or simultaneous administration;

Compound (I) and Compound (II) formulated separately but held in the same pack or device, for sequential or simultaneous administration; and

Compound (I) and Compound (II) in a blended formulation for simultaneous administration.

In each case, each of Compound (I) and/or Compound (II) may be formulated with or without excipients.

Should a third active agent be included, as in a triple combination:

wherein Compound (I), Compound (II) and Compound (III) are presented separately for sequential or simultaneous administration; or

Compound (I), Compound (II) and Compound (III) formulated separately but held in the same pack or device, for sequential or simultaneous administration; or

Compound (I) and Compound (II) are in a blended formulation for simultaneous administration, and Compound (III) is presented separately for either sequential or simultaneous administration; or

Compound (I) and Compound (III) are in a blended formulation for simultaneous administration, and Compound (II) is presented separately for either sequential or simultaneous administration; or

Compound (II) and Compound (III) are in a blended formulation for simultaneous administration, and Compound (I) is presented separately for either sequential or simultaneous administration.

In each case, each of Compound (I) and/or Compound (II), and/or Compound (III) may be formulated with or without excipients.

The present invention further provides a pharmaceutical formulation comprising a combination of Compound (I) and Compound (II) wherein at least one of Compound (I) and Compound (II) is formulated with a pharmaceutically acceptable carrier or excipient.

As generally used, fluticasone propionate would be present in an amount of 250 mcg/dose, whether alone with the bromide compound or in combination with the salmeterol xinafoate. As generally used, salmeterol xinafoate would be present in an amount of 25 or 50 mcg/dose, whether alone with the bromide compound or in combination with fluticasone propionate.

It will be appreciated that for embodiments involving two or more therapeutic agents, the method for the prophylaxis or treatment of a clinical condition in a mammal may include simultaneously, sequentially, or separately administering the various therapeutic agents to the mammal.

The invention also provides a method of preparing a pharmaceutical product as defined herein,

the method comprising either

(a) preparing a separate pharmaceutical composition for administration of the individual compounds of the combination either sequentially or simultaneously, or

(b) preparing a separate pharmaceutical composition for administration of the one of the individual compounds of the combination for either sequentially or simultaneous administration with a separate pharmaceutical composition of the other one or two compounds admixed together, or

(c) preparing a combined pharmaceutical composition for administration of the individual compounds together in the combination for simultaneously use, wherein the pharmaceutical composition comprises the combination together with one or more pharmaceutically acceptable carriers and/or excipients.

It is also recognized that the anticholinergic bromide compound may alternatively be administered in combination with other desirable therapeutic agents, such as other β₂ adrenoreceptor agonists, anti-histamines, and anti-allergic or anti-inflammatory agents. Thus, the present invention also comprises the novel combination of

a) (Endo)-3-(2-cyano-2,2-diphenyl-ethyl)-8,8-dimethyl-8-azonia-bicyclo[3.2.1]octane bromide; and

b) at least one of the following other therapeutic agents selected from the group consisting of short acting and long acting beta 2 agonists: salbutamol, biltolterol, pirbuterol, formoterol, salmefamol, fenoterol or terbutaline, or a salt thereof (e.g. pharmaceutically acceptable salt thereof), for example the sulphate salt or free base of salbutamol or the fumarate salt of formoterol, and wherein the therapeutic agents are optionally present in enantiomerically pure form or as a racemic mixture, as a pharmaceutical product.

Another embodiment of the invention also comprises the novel combination of

a) (Endo)-3-(2-cyano-2,2-diphenyl-ethyl)-8,8-dimethyl-8-azonia-bicyclo[3.2.1]octane bromide; and

b) at least one of the following other therapeutic agents selected from the group consisting of a corticosteroid: beclomethasone, beclomethasone 17-propionate ester, beclomethasone 17,21-dipropionate ester, dexamethasone or an ester thereof, triamcinolone, mometasone or an ester thereof, such as mometasone furoate, ciclesonide, budesonide, flunisolide, or a pharmaceutically acceptable salt thereof.

Suitably, the corticosteroids are selected from beclomethasone, budesonide, flunisolide, mometasone and triamcinolone, and pharmaceutically acceptable salts or esters thereof.

Another embodiment of the invention comprises the novel combination of

a) (Endo)-3-(2-cyano-2,2-diphenyl-ethyl)-8,8-dimethyl-8-azonia-bicyclo[3.2.1]octane bromide; and

b) at least one of the following other therapeutic agents selected from the group consisting of beclomethasone, budesonide, ciclesonide, flunisolide, mometasone, triamcinolone, salbutamol, salbutamol sulphate, biltolterol, pirbuterol, formoterol, formoterol fumarate, and terbutaline, and wherein the therapeutic agents are optionally present in enantiomerically pure form or as a racemic mixture, as a pharmaceutical product.

Another embodiment of the invention comprises the novel combination of

a) (Endo)-3-(2-cyano-2,2-diphenyl-ethyl)-8,8-dimethyl-8-azonia-bicyclo[3.2.1]octane bromide; and

b) at least one of the following other therapeutic agents selected from the group consisting of salmeterol xinafoate, fluticasone propionate, fluticasone furoate, beclomethasone, budesonide, ciclesonide, flunisolide, mometasone, triamcinolone, salbutamol, salbutamol sulphate, biltolterol, pirbuterol, formoterol, formoterol fumarate, and terbutaline, and wherein the therapeutic agents are optionally present in enantiomerically pure form or as a racemic mixture, as a pharmaceutical product.

Mometasone furoate is presently marketed as a nasal spray (Nasonex®), wherein the metered spray containing 100 mg of suspension containing mometasone furoate monohydrate equivalent to 50 μg of mometasone furoate calculated on the anhydrous basis. Mometasone as an inhaled powder is marketed as ASMANEX TWISTHALER® at doses of 220 microgram (mcg) for once daily treatment.

Fluticasone furoate is commercially administered intranasally (Veramyst®), once daily for the treatment of symptoms of seasonal allergic rhinitis and perennial allergic rhinitis. Suitable doses for administration of fluticasone furoate, in combination with Compound (I), may range from about 20 mcg to about 2000 mcg, administered once or twice daily. In another embodiment the doses for administration may be from about 20 mcg to about 500 mcg, or from about 50 to about 1000 mcg/dose, administered once or twice daily. In another embodiment, the dose may be about 110 mcg, administered once or twice daily. It is recognized that the combination of fluticasone furoate and Compound (I) may also include yet a third active agent as is defined herein.

Thus the present invention further provides for a pharmaceutical product comprising any one of the noted combinations of therapeutic agents herein, as a combined preparation for simultaneous, separate or sequential use in the treatment of conditions for which administration of one or more of the therapeutic agents is/are indicated.

Of the suitable combinations for use herein with Compound (I) include: salmeterol, and ciclesonide;

salmeterol, and budesonide;

salmeterol and fluticasone furoate;

salmeterol and mometasone;

formoterol, and budesonide;

formoterol, and ciclesonide;

formoterol and fluticasone propionate;

formoterol and fluticasone furoate;

salbutamol, and beclomethasone;

salbutamol, and budesonide;

salbutamol, and fluticasone furoate;

terbutaline, and fluticasone propionate; and

terbutaline, and fluticasone furoate.

As a number of these combinations are already commercially available, and not wishing to be limited solely to those fixed combination dosages on the market place, the following information is provided.

Chiesi's (FOSTER; INUVAIR), is a fixed combination of the beta2 adrenergic receptor agonist, formoterol, and the corticosteroid, beclometasone. This dosage form includes 100 mcg beclomethasone and 6 mcg formoterol.

Ciclesonide (Omnaris®/Alvesco®) is commercially available as an inhaled corticosteroid, at 160 and 320 mcg/dose twice daily. It can, however be administered in 100-1600 microgram/day doses.

Budesonide is available as Pulmicort Turbuhaler®, or as a nasal inhalant (Rhinocort® Aqua®). Some dosages for Rhinocort in Children ≧6 years and Adults: 64 mcg/day as a single 32 mcg spray in each nostril. With 0.5 to 1 mg/day as an approved dosage. For oral inhalation doses may be 200 or 400 mcg twice daily in adults.

Albuterol (salbutamol sulphate) is commercially available in a number of forms, such as Proventil®, ProAir HFA® or VoSpire ER®. For Proventil, each actuation delivers 120 mcg albuterol sulfate, USP from the valve and 108 mcg albuterol sulfate, USP from the mouthpiece (equivalent to 90 mcg of albuterol base from the mouthpiece).

Fluticasone propionate is available as Flovent Diskus® in 50 mg, 100 mg and 250 mg dosages, or as Flovent HFA®.

Schering Plough/Novartis are developing a mometasone furoate and formoterol fumarate fixed combination dose 400/10 mcg and 200/10 mcg at twice daily dosing (bid). Formoterol fumerate (or fumarate) is marketed separately as Foradil® Aerolizer by Norvatis and contains 12 mcg of the formoterol and 25 mg of lactose as a carrier, for twice daily administration.

There is a commercially available product of formoterol fumarate and budesonide available as AstraZeneca's Symbicort® containing budesonide at 80 mcg and formoterol at 4.5 mcg and budesonide at 160 mcg and 4.5 mcg formoterol.

Two dose combinations of FLUTIFORM®, in development are thought to contain 100 mcg fluticasone and 10 mcg formoterol, or 250 mcg fluticasone and 10 mcg formoterol.

High daily doses of inhaled corticosteroids are generally thought to be:

>1000 mcg beclomethasone in a chlorofluorocarbon (CFC) dosage form

>500 mcg beclomethasone in a hydrofluoroalkane (HFA) dosage form

>1000 mcg budesonide in a dry powder inhaler (DPI)

>2000 mcg flunisolide

>500 mcg fluticasone

>400 mcg Mometasone Furoate

>2000 mcg triamcinolone acetonide

Another embodiment of the present invention comprises the novel combination of

a) (Endo)-3-(2-cyano-2,2-diphenyl-ethyl)-8,8-dimethyl-8-azonia-bicyclo[3.2.1]octane bromide; and

b) at least one of the following other therapeutic agents selected from the group consisting of an anti-histamine where suitable for inhalation, such as methapyrilene, cetirizine, loratadine, desloratadine or fexofenadine.

It will also be appreciated from the above, that similar to the combinations discussed herein, these respective therapeutic agents for the combined preparations can be administered simultaneously, either in the same or different pharmaceutical formulations, or separately or sequentially. If there is separate or sequential administration, it will also be appreciated that the subsequently administered therapeutic agents should be administered to a patient within a time scale so as to achieve, or more particularly optimise, the above referred to advantageous synergistic therapeutic effect of a combined preparation as present in a pharmaceutical product according to the present invention.

### Synthetic Chemistry

(Endo)-3-(2-cyano-2,2-diphenyl-ethyl)-8,8-dimethyl-8-azonia-bicyclo[3.2.1]-octane bromide is described in WO2005/037280, and may be made as described below. Suitably pharmaceutically acceptable anions as the quaternary salt, such as the bromide or iodo salts may be made using 3-((Endo)-8-methyl-8-aza-bicyclo[3.2.1]oct-3-yl)-2,2-diphenyl-propionitrile as a chemical intermediate with a suitable methyl anion, such as methyl bromide, or methyl iodide, etc.

### Example 1

### 3-((Endo)-8-methyl-8-aza-bicyclo[3.2.1]oct-3-yl)-2,2-diphenyl-propionitrile, TFA salt

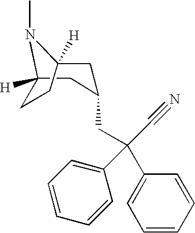

a) Preparation of ((endo)-8-methyl-8-aza-bicyclo[3.2.1]oct-3-yl)-methanol

A mixture of 1,1-dimethylethyl(endo)-3-(hydroxymethyl)-8-azabicyclo[3.2.1]octane-8-carboxylate (0.50 g, 2.05 mmol) and LiAlH₄ (6.16 mL, 1.0 M in THF, 6.16 mmol) was heated at 80° C. with a microwave reactor for 60 min. The solution was then mixed with saturated Na₂SO₄ solution, ethyl ether was added, dried over potassium carbonate and filtered through celite and concentrated to afford the title compound (0.31 g, 97%): LCMS (ES) m/z 156 (M+H)⁺; ¹H-NMR (CDCl₃) δ 1.28 (s, 1H), 1.59 (m, 4H), 1.90 (m, 1H), 2.13 (m, 4H), 2.32 (s, 3H), 3.17 (s, 2H), 3.59 (d, 2H).

b) Preparation of (endo)-3-iodomethyl-8-methyl-8-aza-bicyclo[3.2.1]octane

A solution of iodine (6.67 g, 25.8 mmol) and ((endo)-8-methyl-8-aza-bicyclo[3.2.1]oct-3-yl)-methanol (2.0 g, 12.9 mmol) in CH₂Cl₂ (120 mL) was mixed with PPh₃ (on resin, 8.6 g, 3 mmol/g, 25.8 mmol). The resultant mixture was stirred for 17 hours, filtered and concentrated to afford the title compound (2.63 g, 77%): LCMS (ES) m/z 266 (M+H)⁺; ¹H-NMR (CDCl₃) δ 2.05 (m, 4H), 2.39 (m, 3H), 2.79 (d, 3H), 2.98 (m, 2H), 3.45 (d, 2H), 3.81 (s, 2H).

c) Preparation of 3-((endo)-8-methyl-8-aza-bicyclo[3.2.1]oct-3-yl)-2,2-diphenyl-propionitrile, TFA salt

To a solution of Ph₂CHCN (2.32 g, 12.0 mmol) in DMF (20 mL) was added NaH (0.288 g, 12.0 mmol). After stirring for 10 seconds, (3-endo)-3-iodomethyl-8-methyl-8-aza-bicyclo[3.2.1]octane (1.06 g, 4.0 mmol) was added and the resultant mixture was stirred at room temperature for 60 minutes. The mixture was worked up with 5 ml DMSO and 1 ml HCL (2N) then filtered. Further purification by reverse phase HPLC (Gilson), with TFA afforded the title compound (1.16 g, 93%): LCMS (ES) m/z 331 (M+H)⁺; ¹H-NMR (CDCl₃) δ 1.64 (m, 2H), 2.14 (m, 1H), 2.26 (m, 2H), 2.34 (m, 2H), 2.52 (m, 2H), 2.75 (m, 5H), 3.83 (s, 2H), 7.39 (d, 10H).

### Example 2

### (Endo)-3-(2-cyano-2,2-diphenyl-ethyl)-8,8-dimethyl-8-azonia-bicyclo[3.2.1]octane bromide

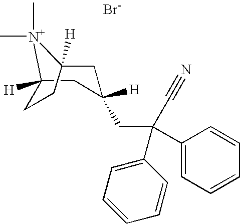

A solution of 3-((endo)-8-methyl-8-aza-bicyclo[3.2.1]oct-3-yl)-2,2-diphenyl-propionitrile, TFA (310 mg, 0.938 mmol) was washed with potassium carbonate in acetonitrile and stirred for 20 minutes. After concentration under vacuum, the residue in acetone (6.0 mL) was mixed with MeBr (4.69 mL, 2.0 M in t-BuOMe, 9.38 mmol). The resultant mixture was stirred at room temperature for about 90 minutes, filtered and concentrated. The residue was washed with acetone (2×3 mL) to afford the title compound (333 mg, 83%): LCMS (ES) m/z 345 (M)⁺; ¹H-NMR (MeOD) δ 1.82 (d, 2H), 2.17 (m, 1H), 2.35 (m, 2H), 2.49 (m, 4H), 3.01 (d, 2H), 3.07 (s, 3H), 3.10 (s, 3H), 3.79 (s, 2H), 7.36 (m, 2H), 7.43 (m, 4H), 7.49 (m, 4H).

### Biological Examples

In a human clinical trial, the bromide compound was found to be an effective long-acting anti-muscarinic bronchodilator having an unexpectantly faster onset of action than tiotropium. This demonstration of improved onset of action and the dose dependent nature of the bromide compound provides for both a twice daily (bd) and once daily (qd) dosing alone or depending upon the secondary therapeutic agent it is administered with.

The bromide salt is a high-affinity, pan-active, competitive and reversible long-acting muscarinic receptor also demonstrating in vitro, the onset half times to 50% inhibition of carbachol-induced contraction of isolated human bronchial strips for the bromide compound of 25 min, ipratropium at 10 min, and tiotropium at 10 min when compared at single concentration of 10 nM. At the same concentration, the offset half time for the bromide compound of 195 min was longer than that of ipratropium (24 min) and shorter than that of tiotropium (314 min).

In another clinical trial using a binary mixture of the bromide compound and lactose monohydrate, the safety, tolerability, pharmacodynamics and pharmacokinetics of single escalating doses of the bromide compound (20, 60, 100, 250 and 350 mcg via the Diskus), and tiotropium (18 mcg) in 20 healthy male ipratropium-responsive subjects were compared.

All doses of the bromide compound and tiotropium resulted in statistically significantly greater increases in airway conductance (sGaw) values than placebo. All bromide compound treatment groups showed increases compared with placebo of between 36% and 49% at 12 and 24 hours, except for the 20 mcg dose which demonstrated a 16% increase after 24 hours; the differences were similar in magnitude to those shown for tiotropium, with the exception of the 20 mcg dose at 24 hours.

A third clinical study assessed the safety, tolerability, pharmacodynamics and pharmacokinetics of single inhaled doses of the bromide compound (20, 50 and 100 mcg via Diskus), and tiotropium (18 mcg) in 31 male and female ipratropium-responsive COPD subjects.

The Mean FEV₁ values for all bromide compound doses were statistically significantly higher than placebo at each time point assessed over 24 hours. Mean FEV₁ values peaked at 2 hours and decreased consistently to 24 hours for each dose. For the 20, 50 mcg and 100 mcg doses, adjusted mean differences versus placebo at 12 hours were 241 mL, 288 mL and 305 mL respectively; these reduced to 135 and 136 mL at 24 hours in the case of 20 mcg and 50 mcg doses, but remained above 200 mL (comparable to tiotropium) in the case of the 100 mcg dose, indicating a dose-related effect on bronchodilation and duration of action.

In a fourth clinical trial, which was multi-centre, partially blinded, 3-way crossover, having an incomplete block design for COPD patients, 5 treatment arms were studied: Salmeterol 50 mcg plus the bromide compound at 20 mcg and 50 mcg (twice daily), salmeterol 50 mcg alone (twice daily), tiotropium 18 mcg (once daily) and placebo. The study involved a 2-week run-in; 3 treatment periods of 7 days, each separated by 14-day washout; and 2-week follow up.

Primary endpoints were used to estimate the bronchodilatory effect of the bromide compound (20 mcg and 50 mcg bid) administered concurrently with salmeterol 50 mcg bid, as compared with placebo

Secondary endpoints were to estimate the bronchodilatory effect of salmeterol and tiotropium compared with placebo, and with concurrent treatment with the bromide compound (20 mcg and 50 mcg) plus salmeterol.

A primary marker was the morning trough FEV1 on Day 8.

A secondary marker was the morning trough FEV1 on Day 2; Trough FVC on Days 2 and 8; Post-dose serial FEV1 and FVC over 24 hours; Trough sGaw, Raw, IC and RV on Days 2 and 8, and post-dose serial measurements over 25 hours; plasma concentrations and derived PK parameters for the bromide compound and salmeterol.

Both combination treatments (bromide compound 20 mcg+salmeterol, bromide compound 50 mcg+salmeterol) showed improvements in lung function (FEV1, FVC, sGAW) as compared with placebo, salmeterol and tiotropium.

The trough FEV1 on Day 8 on both combination treatments was unexpectantly more than 200 mL higher than placebo, more than 100 mL higher than salmeterol and more than 80 mL higher than tiotropium.

Use of rescue VENTOLIN was reduced on the combination treatments compared with placebo, salmeterol and tiotropium.

No obvious dose response was observed between the two combination treatments.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

1. A pharmaceutical product comprising
a) a first therapeutic agent which is 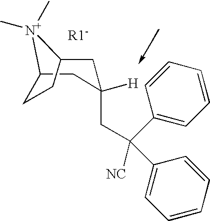
wherein:
the H atom indicated is in the exo position;
R1⁻ represents a pharmaceutically acceptable anion; and
b) at least one of the following other therapeutic agents selected from the group consisting of salmeterol xinafoate and fluticasone propionate; and wherein the therapeutic agents are optionally present in enantiomerically pure form or as a racemic mixture.

2. The product according to claim 1 wherein the first therapeutic agent is (Endo)-3-(2-cyano-2,2-diphenyl-ethyl)-8,8-dimethyl-8-azonia-bicyclo[3.2.1]octane bromide.

3. The product according to claim 1 wherein the first therapeutic agent and the at least one other therapeutic agent is prepared for administration as an admixture or as separate compositions.

4. The product according to claim 2 which comprises the first therapeutic agent and salmeterol xinafoate is present in an amount of 50 mcg/dose.

5. (canceled)

6. The product according to claim 1 wherein the first therapeutic agent is present in an amount of about 10 to 100 mcg/dose.

7. The product according to claim 1 wherein the first therapeutic agent and salmeterol xinafoate are present as separate compositions.

8. The product according to claim 1 wherein the first therapeutic agent and salmeterol xinafoate are in admixture with each other.

9. The product according to claim 1 which comprises the first therapeutic agent and fluticasone propionate present in an amount of 250 mcg/dose.

10.-11. (canceled)

12. The product according to claim 10 wherein the first therapeutic agent and fluticasone propionate are present as separate compositions.

13. The product according to claim 10 wherein the first therapeutic agent and fluticasone propionate are in admixture with each other.

14. The product according to claim 1 wherein both salmeterol xinafoate and fluticasone propionate are present.

15. The product according to claim 14 wherein the first therapeutic agent is in a separate composition from the compositions of salmeterol xinafoate and fluticasone propionate.

16. The product according to claim 14 wherein the salmeterol xinafoate and fluticasone propionate are each in independent compositions.

17. The product according to claim 14 wherein the salmeterol xinafoate and fluticasone propionate are in admixture with each other.

18. The product according to claim 14 wherein the first therapeutic agent is present as an admixture with salmeterol xinafoate and is separate from the fluticasone propionate.

19. The product according to claim 14 wherein the first therapeutic agent is present as an admixture with fluticasone propionate and is a separate from the salmeterol xinafoate.

20.-22. (canceled)

23. The product according to claim 1 in a form suitable for administration by oral or nasal inhalation.

24. The product according to claim 23 in the form of an aerosol formulation.

25. The product according to claim 24, which further comprises a propellant selected from the group consisting of 1,1,1,2-tetrafluoroethane, 1,1,1,2,3,3,3-heptafluoropropane, monofluorotrichloromethane and dichlorodifluoromethane, or any mixture of two or more thereof.

26. The product according to claim 25 further comprising a co-solvent.

27. The product according to claim 24 further comprising a surface-active agent.

28. The product according to claim 23 wherein the form is suitable for administration by inhalation is via a medicament dispenser selected from a reservoir dry powder inhaler, a unit-dose dry powder inhaler, a pre-metered multi-dose dry powder inhaler, a nasal inhaler or a pressurized metered dose inhaler.

29. The product according to claim 28 which is a pressurized metered dose inhaler.

30. The product according to claim 1 in the form of an inhalation powder.

31. The product according to claim 30 which further comprises lactose as a pharmaceutically acceptable excipient.

32. The product according to claim 28 which is a dry powder inhaler containing a composition according to claim 1.

33. The product according to claim 1 in the form of a propellant free inhalation solution or suspension.

34-37. (canceled)

38. A method for the prophylaxis or treatment of inflammatory or respiratory tract diseases, comprising administering either sequentially or simultaneously, to a patient in need thereof, a product according to claim 1 comprising a first therapeutic agent present in an amount of about 10 to 100 mcg/dose and at least one other therapeutic agent.

39. The method according to claim 38 wherein the disease is selected from the group consisting of chronic obstructive lung disease, chronic bronchitis, asthma, chronic respiratory obstruction, pulmonary fibrosis, pulmonary emphysema and allergic rhinitis.

40. The method according to claim 39 for the treatment of asthma and/or chronic obstructive pulmonary disease (COPD), by simultaneous or successive administration.

41. The method according to claim 38 wherein administration is via inhalation by the mouth or nose.

42. The method according to claim 41 wherein administration is via a medicament dispenser selected from a reservoir dry powder inhaler, a pre-metered multi-dose dry powder inhaler, a nasal inhaler or a pressurized metered dose inhaler.

43. The method according to claim 42 wherein the first therapeutic agent is administered to a human in a pre-metered multi-dose dry powder inhaler, and wherein the first therapeutic agent and the at least one other therapeutic agent is stored in the inhaler in a separate composition.

44. The method according to claim 43 wherein the other therapeutic agent is salmeterol xinafoate present in an amount of 50 mcg/dose.

45. The method according to claim 44 which comprises a second other therapeutic agent which is fluticasone propionate present in an amount of 250 mcg/dose.

46. The method according to claim 45 wherein both salmeterol xinofoate, and fluticasone propionate are present and are in admixture with each other, separate from the first therapeutic agent.

47.-49. (canceled)

50. The method according to claim 38 in a form suitable for once or twice daily administration.

51. The method according to claim 50 in the form of an aerosol.

52. A pharmaceutically acceptable composition comprising a pharmaceutically acceptable anion of (Endo)-3-(2-cyano-2,2-diphenyl-ethyl)-8,8-dimethyl-8-azonia-bicyclo[3.2.1]octane in admixture with a ternary agent selected from cellobiose octaacetate, calcium stearate or magnesium stearate.

53. The composition according to claim 52 wherein the anion is bromide or iodide and the ternary agent is magnesium stearate.

54. A pharmaceutically acceptable composition comprising a pharmaceutically acceptable anion of (Endo)-3-(2-cyano-2,2-diphenyl-ethyl)-8,8-dimethyl-8-azonia-bicyclo[3.2.1]octane in admixture with at least one carrier which is lactose.

55. The composition according to claim 54 which further comprises a ternary agent which is magnesium stearate.

56. A product according to claim 1 wherein the first therapeutic agent and the at least one of other therapeutic agent is formulated with at least one pharmaceutically acceptable carrier or excipient.

57. A product according to claim 56 wherein the at least one carrier is lactose.

58. A product according to claim 56 wherein at least one of the therapeutic agents is formulated with a ternary agent.

59. A product according to claim 58 wherein the ternary agent is magnesium stearate.

60. The product according to claim 1 wherein the pharmaceutically anion is selected from chloride, bromide, iodide, sulfate, benzene sulfonate or toluene sulfonate.
